(19) United States
(12) Patent Application Publication
(10) Pub. No.: US 2018/0043110 A1
(43) Pub. Date: Feb. 15, 2018

(54) SYRINGE PLUNGER AND PRESSURE INDICATOR

(71) Applicant: OWEN MUMFORD LIMITED, Oxfordshire (GB)

(72) Inventor: Damian Alexander HOLLAND, Oxfordshire (GB)

(21) Appl. No.: 15/557,690

(22) PCT Filed: Mar. 14, 2016
(86) PCT No.: PCT/GB2016/050694

§ 371 Date: Sep. 12, 2017

(30) Foreign Application Priority Data
Mar. 13, 2015 (GB) 1504319.3

(51) Int. Cl. A61M 5/48; A61M 5/315

(52) U.S. Cl. CPC A61M 5/486; A61M 5/31515; A61M 2205/582

(57) ABSTRACT

A syringe plunger for insertion within a barrel of a syringe for expelling the contents of the barrel from an open end thereof and a pressure indicator for fitting to a syringe plunger. The syringe plunger and pressure indicator comprising: a first portion; a second portion moveable with respect to the first portion if a relative force between the first portion and the second portion exceeds a threshold; and an indicator, wherein translation of movement of the first portion relative to the second portion causes mechanical movement of the indicator.

### TECHNICAL FIELD

The invention relates to syringes and syringe plungers. More specifically, the invention relates to, but is not limited to, syringes and syringe plungers for monitoring a pressure at which a substance is expelled from a syringe.

### BACKGROUND

The pressure at which a fluid is expelled from a syringe may have significant consequences on the efficacy of the fluid to undertake the task it was designed for. For example, where a medicament is expelled from a syringe into a human or animal subject, if too high a pressure is used then this may damage the medicament such that it is less effective or non-effective. In addition, if a fluid is expelled from a syringe into a human or animal subject at too high a pressure then this may cause significant discomfort for the subject. The acceptable pressure for a given fluid may alter. The acceptable pressure for a given fluid may be dependent on the viscosity of the fluid.

Apparatus exist that use a power source and an indicator source that come into contact and emit a beep or colour change, such as U.S. Pat. No. 5,270,685.

### SUMMARY

According to the invention in a first aspect, there is provided a syringe plunger for insertion within a barrel of a syringe for expelling the contents of the barrel from an open end thereof, the syringe plunger comprising: a first portion; a second portion moveable with respect to the first portion if a relative force between the first portion and the second portion exceeds a threshold; and an indicator, wherein translation of movement of the first portion relative to the second portion causes mechanical movement of the indicator.

Optionally, the first portion comprises a plunger head and the second portion comprises a plunger shaft.

Optionally, the syringe plunger further comprises a resiliently deformable member coupled to the first and second portions.

Optionally, the resiliently deformable member is configured to bias the first portion away from the second portion and/or bias the second portion away from the first portion.

Optionally, the resiliently deformable member is positioned between the first portion and the second portion and is configured to compress if the relative force between the first portion and the second portion exceeds the threshold.

Optionally, the resiliently deformable member is configured to bias the first portion towards the second portion and/or bias the second portion towards the first portion.

Optionally, the resiliently deformable member is positioned between the first portion and the second portion and is configured to expand if the relative force between the first portion and the second portion exceeds the threshold.

Optionally, the resiliently deformable member comprises one or more arms extending between the first portion and the second portion.

Optionally, the one or more arms are configured to flex with respect to the syringe plunger on movement of the first portion with respect to the second portion.

Optionally, the indicator is configured to provide a haptic indication to a user of a syringe.

Optionally, the indicator comprises an indicator rod configured to contact the user's skin after movement of the first portion with respect to the second portion.

Optionally, the indicator rod is fixed with respect to the second portion.

Optionally, the first portion comprises an aperture in a force application surface, and wherein the indicator rod is configured to pass through the aperture after movement of the first portion towards the second portion.

According to the invention in a second aspect, there is provided a pressure indicator for coupling to a syringe plunger, the pressure indicator comprising: a first portion; a second portion moveable with respect to the first portion if a relative force between the first portion and the second portion exceeds a threshold; and an indicator, wherein translation of movement of the first portion relative to the second portion causes mechanical movement of the indicator.

Optionally, the first portion comprises a plunger head and the second portion is configured to be directly coupled to a syringe plunger.

Optionally, the pressure indicator further comprises a resiliently deformable member coupled to the first and second portions.

Optionally, the resiliently deformable member is configured to bias the first portion away from the second portion and/or bias the second portion away from the first portion.

Optionally, the resiliently deformable member is positioned between the first portion and the second portion and is configured to compress if the relative force between the first portion and the second portion exceeds the threshold.

Optionally, the resiliently deformable member is configured to bias the first portion towards the second portion and/or bias the second portion towards the first portion.

Optionally, the resiliently deformable member is positioned between the first portion and the second portion and is configured to expand if the relative force between the first portion and the second portion exceeds the threshold.

Optionally, the resiliently deformable member comprises one or more arms extending between the first portion and the second portion.

Optionally, the one or more arms are configured to flex with respect to the syringe plunger on movement of the first portion with respect to the second portion.

Optionally, the indicator is configured to provide a haptic indication to a user of a syringe.

Optionally, the indicator comprises an indicator rod configured to contact the user's skin after movement of the first portion with respect to the second portion.

Optionally, the indicator rod is fixed with respect to the second portion.

Optionally, the first portion comprises an aperture in a force application surface, and wherein the indicator rod is configured to pass through the aperture after movement of the first portion with respect to the second portion.

According to the invention in a third aspect, there is provided a syringe plunger comprising a pressure indicator described above.

According to the invention in a fourth aspect, there is provided a syringe comprising a barrel and a syringe plunger as described above.

According to the invention in a fifth aspect, there is provided a kit of parts comprising: a first portion of a syringe plunger; a second portion of a syringe plunger; and an indicator, wherein the first portion is configured to be coupled to the second portion such that it is moveable with respect to the second portion if a relative force between the first portion and the second portion exceeds a threshold, and wherein translation of movement of the first portion relative to the second portion causes mechanical movement of the indicator.

Optionally, the indicator forms part of one of the first portion and the second portion.

Optionally, the kit of parts further comprises a resiliently deformable member coupled to the first and second portions.

Optionally, the resiliently deformable member forms part of the first portion and/or the second portion.

### BRIEF DESCRIPTION OF THE FIGURES

FIGS. 1a-b show perspective views of a syringe plunger;

FIG. 2a shows a side elevation of a syringe plunger in an extended state;

FIG. 2b shows a section through a syringe plunger in an extended state;

FIG. 3a shows a side elevation of a syringe plunger in a compressed state;

FIG. 3b shows a section through a syringe plunger in a compressed state;

FIGS. 4a-b show perspective views of a pressure monitor for fitting to a syringe plunger;

FIG. 5a shows a side elevation of a pressure monitor in an extended state;

FIG. 5b shows a section through a pressure monitor in an extended state;

FIG. 6a shows a side elevation of a pressure monitor in a compressed state;

FIG. 6b shows a section through a pressure monitor in a compressed state;

FIGS. 7a-b show perspective views of a pressure monitor for fitting to a syringe plunger;

FIG. 8a shows a side elevation of a pressure monitor in an extended state;

FIG. 8b shows a section through a pressure monitor in an extended state;

FIG. 9a shows a side elevation of a pressure monitor in a compressed state;

FIG. 9b shows a section through a pressure monitor in a compressed state; and

FIG. 10a-b show perspective views of a syringe comprising a syringe plunger.

### DETAILED DESCRIPTION

Generally disclosed herein are methods and apparatus for monitoring pressure within a barrel of a syringe. The pressure within the barrel typically increases as a user depresses a syringe plunger. The methods and apparatus disclosed comprise means to indicate to the user if the pressure within the barrel exceeds a threshold value.

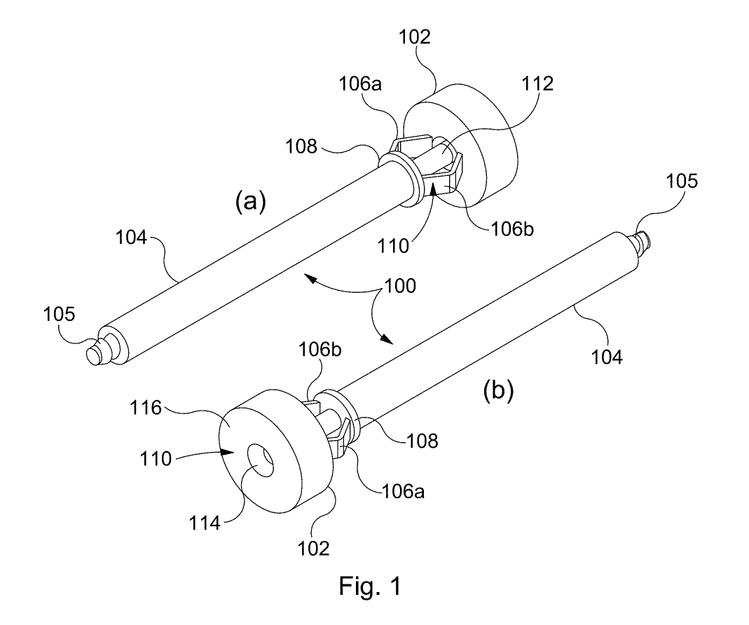

Referring to FIGS. 1a and 1b, a syringe plunger 100 is shown. The syringe plunger 100 comprises a first portion 102, which may be a plunger head, and a second portion 104, which may be a plunger shaft. The terms plunger head and plunger shaft are used throughout this document, but it should be understood that the first and second portions 102, 104 of the syringe plunger 100 may comprise other features. For example, the separation between the first and second portions 102, 104 may be at a point along the plunger shaft.

The plunger head 102 is moveable with respect to the plunger shaft 104 if a relative force between the plunger head 12 and the plunger shaft 104 exceeds a threshold value. That is, if a first force is applied to the plunger head 102, say from a user's finger or thumb while operating a syringe, and a second force is applied to the plunger shaft, say due to an increase in pressure inside a barrel of the syringe, if the resultant force exceeds a threshold value then the plunger head 102 will move with respect to the plunger shaft 104. In exemplary syringe plungers 100, the plunger head 102 moves towards the plunger shaft 104.

The plunger shaft 104 in FIGS. 1-3 has a substantially circular cross section, although other cross sections are possible. For example, the plunger shaft 104 may have a cross shaped cross section. In addition, the plunger shaft may comprise a bung connector 105 for connection to a bung that travels in the barrel of the syringe to expel the contents of the syringe from an open end of the barrel. In exemplary syringe plungers, the bung connector 105 may comprise a threaded portion for securing the bung to the syringe plunger 100. In other arrangements, the bung may be connected to the syringe plunger as part of the manufacturing process, or the plunger shaft 104 may butt up against the bung without any connection of the plunger shaft 104 to the bung.

In the exemplary syringe plunger of FIGS. 1-3, at least one resiliently deformable member is positioned between the plunger head 102 and the plunger shaft 104. However, the resiliently deformable member is optional, as other means may be employed to provide a coupling between the plunger head 102 and the plunger shaft 104 allowing movement of the plunger head 102 towards the plunger shaft 104 if the relative force between them exceeds a threshold. For example, there may be a friction engagement between the plunger head 102 and the plunger shaft 104 that is configured to be overcome if the relative force exceeds a threshold. Alternatively, a ratchet and pawl arrangement may be used. Other options are available that are not elaborated on here.

In the exemplary syringe plunger of FIGS. 1-3, the resiliently deformable member comprises a plurality of arms 106a-b. The arms 106a-b are positioned between the plunger head 102 and the plunger shaft 104. In exemplary syringe plungers 100, the arms 106a-b are connected to the plunger head 102 and/or the plunger shaft 104. In other exemplary syringe plungers 100, the arms 106a-b may form part of the plunger head 102 and/or the plunger shaft 104. The plunger shaft 104 comprises a lip 108 at a head end thereof. The lip provides a top surface of the plunger shaft 104 that the resilient member may interact with and/or be connected to.

The arms 106a-b are configured to bow or flex outwardly with respect to a longitudinal axis of the syringe plunger 100 on movement of the plunger head 102 towards the plunger shaft 104. In the exemplary syringe plunger 100 of FIGS. 1-3, the arms 106a-b comprise first and second portions with a bend in between to form a basic spring. The arms 106a-d have the bend at a halfway point along the entire length of the arm, although this is not essential. In other exemplary syringe plungers, the arms may comprise one continuous portion that is bowed perpendicular to the longitudinal axis between the plunger head 102 and the plunger shaft 104 so as to form a basic spring. The arms are manufactured from a suitable material such that they have sufficient elasticity to return towards their original shape if a relative force between the plunger head 102 and the plunger shaft 104 is reduced below a threshold.

It is noted that the resiliently deformable member may be provided by any other suitable means, such as a helical spring.

The syringe plunger 100 also comprises an indicator 110. In the exemplary syringe plunger 100 of FIGS. 1-3, the indicator comprises an indicator rod 112 and an aperture 114 in the plunger head 102. The indicator rod 112 is configured to pass through the aperture 114 to protrude from an upper (or force application) surface 116 of the plunger head 102 after the plunger head 102 moves towards the plunger shaft 104. That is, the movement of the plunger head 102 causes the aperture 114 to pass down over the indicator rod 112. In this way, the movement of the plunger head 102 is translated into a mechanical actuation or movement of the indicator 110. The indicator rod 114 may be connected to the plunger shaft 104 and, in specific exemplary syringe plungers, may be formed as part of the plunger shaft 104.

In the case of the indicator 110, the movement of the plunger head 102 is mechanically translated into mechanical operation of the indicator 110 and this may also be the case with other exemplary arrangements. The indicator may comprise other features that are operational based on direct or indirect mechanical translation to provide an indication to a user. The indicator 110 provides a haptic response in that the indicator rod is configured to contact a user's skin, probably on the thumb or finger. However, other indicators may provide an audible indication, such as the clicking of a ratchet and pawl, or a visual indication, such as a colour change or the like.

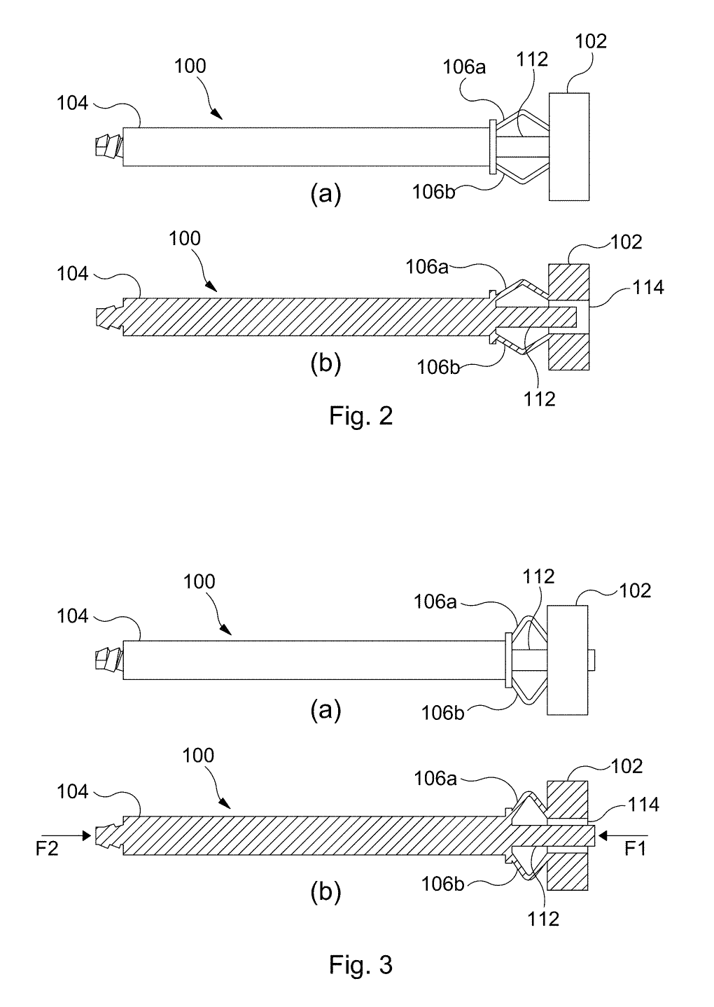

FIGS. 2a and 2b show a side elevation of the syringe plunger 100 and a longitudinal section through the syringe plunger 100, respectively. In FIGS. 2a and 2b, the syringe plunger 100 is in an extended state, in that the plunger head 102 is biased away from the plunger shaft 104 as no force is applied to either.

As a force F1 is applied, most likely by a user's thumb, to the plunger head 102, the syringe plunger 100 will travel within the barrel of a syringe (not shown) to expel the contents of the barrel from the opposite and open end. The open end of the barrel typically has an aperture that has a smaller diameter than the internal diameter of the barrel and the pressure within the barrel therefore increases with the force F1 that is applied to the plunger head 102. This results in a force F2 opposing the force F1. If the resultant force (i.e. the addition of F1 and F2) is greater than a threshold value then the plunger head 102 begins to move towards the plunger shaft 104, as shown in FIGS. 3a and 3b, which show a side elevation of the syringe plunger 100 and a longitudinal section through the syringe plunger 100, respectively. In FIGS. 3a and 3b, the syringe plunger 100 is shown in a compressed state, in that the resultant force has exceeded the threshold value. The threshold value may be determined by the resilient member (e.g. the arms 106a-b) or the other coupling of the plunger head 102 and the plunger shaft 104.

As shown in FIGS. 3a and 3b, the plunger head 102 has moved towards the plunger shaft 104. The arms 106a-b have flexed or bowed outwardly with respect to the syringe plunger 100. The indicator rod 112 has passed through the aperture 114 and protrudes from a surface 116 of the plunger head 102. As such, the indicator rod 114 contacts the skin of the user and thereby produces a haptic indication. As stated previously, other indications, such as audible or visual indications are possible.

In exemplary syringe plungers, the indicator rod 112 may be tapered towards the plunger head 102 such that the haptic indication is more obvious to the user. That is, by reducing the cross sectional area of the indicator rod 112 at the plunger head end, the amount of pressure applied to the user's skin is increased.

If a user increases the force F1 too much, i.e. they press too hard on the plunger head 102, then the indicator rod 112 will contact the skin of the user and indicate that they should reduce the force F1. As the force F1 is reduced, the resiliently deformable member expands towards its resting state and the plunger head 102 moves away from the plunger shaft 104 and the indicator rod 112 recedes back within the aperture 114 and no longer protrudes from the surface 116. This indicates to the user that the force F1 is now below the required force. It is noted that the return of the indicator is not essential for the operation of the syringe plunger.

The haptic response provided by the indicator advantageously provides a steady indication that is not sudden and is not likely to shock the user. This is advantageous as shocks to users of syringes may be dangerous for any subject on which the syringe is being used. In addition, the mechanical translation of the movement of the plunger head 102 with respect to the plunger shaft 104 means that a simple apparatus may be formed. Indeed, in exemplary embodiments, the syringe plunger may be formed of a single piece and may be moulded plastics material. The inventor has appreciated that the mechanical movement of the plunger head may provide the power to operate the indicator. A further advantage is that the indicator may provide an indication to the user that the syringe plunger has completed its travel within the barrel of the syringe and, therefore that all of the fluid contained in the barrel has been expelled from the open end. As the syringe plunger (or the bung) contacts the end of the barrel, the plunger head will begin to move towards the plunger shaft and the indicator will indicate to the user that the syringe plunger has reached the limit of its travel.

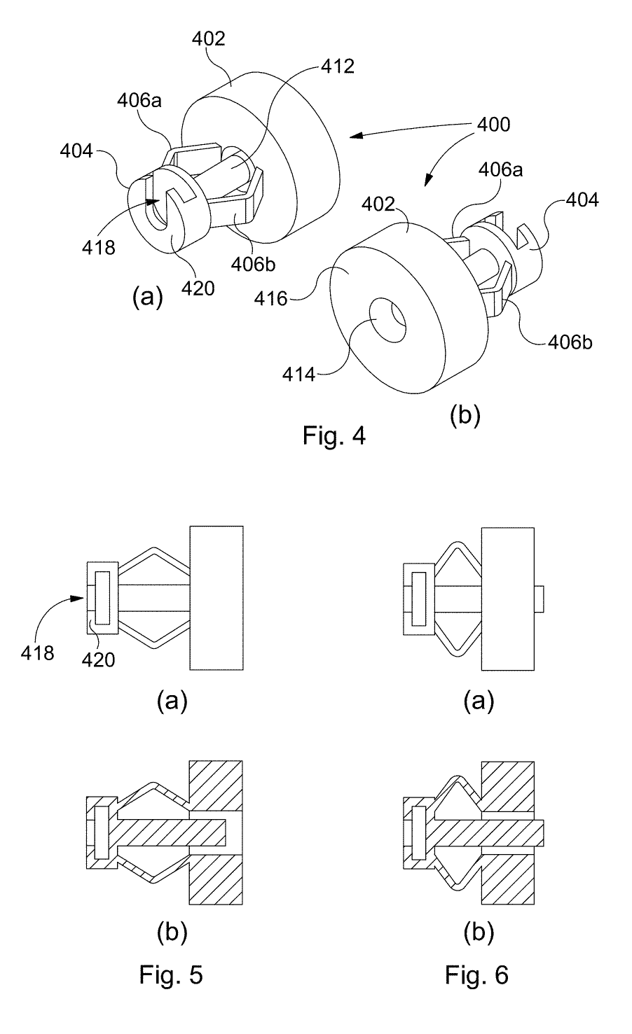

FIGS. 4-9 show exemplary pressure indicators 400, 700 for coupling to a syringe plunger. The pressure indicators 400, 700 comprise the same or similar features as the syringe plunger 100. These have been given corresponding reference numerals and are not explained in detail again here. It is noted that any corresponding features between the syringe plunger 100 and the pressure indicators 400, 700 that are described as optional in respect of the syringe plunger are also optional in respect of the pressure indicators. In addition, the description of the pressure indicators 400, 700 is given in detail with respect to the exemplary pressure indicators 400, 700 shown in FIGS. 4-9, however, the features of the pressure indicators 400, 700 that correspond to the features of the syringe plunger 100 may also be broadly defined as in the description above. For the avoidance of doubt, the description of the syringe plunger 100 above relates equally to the corresponding features of the pressure indicators 400, 700.

The pressure indicators 400, 700 comprise a first portion 402, 702 and a second portion 404, 704. The first portion 402, 702 may be the plunger head and the second portion 404, 704 may be a coupling means that is defined in more detail below. A resiliently deformable member (e.g. arms 406a-b and 706a-b) is positioned between the plunger head 402, 702 and the coupling means 404, 704. An indicator rod 412, 712 is configured to pass through an aperture 414, 714.

The second portion may comprise a coupling means 404, 704 for coupling the pressure indicator 400, 700 to a known syringe plunger. In this way, the pressure indicator may be retro-fitted to an existing syringe plunger.

The coupling means 404 comprises a slot 418 in a retaining wall 420. Syringe plungers typically comprise a shaft and a head, wherein the head has a greater diameter than the shaft such that a lip is formed all around the circumference of the top of the shaft. In the coupling means 404, the shaft of the syringe plunger passes through the slot 418 and the lip formed by the head is held behind the retaining wall 420. There may additionally be a locking means to hold the syringe plunger in a coupled configuration with the coupling means.

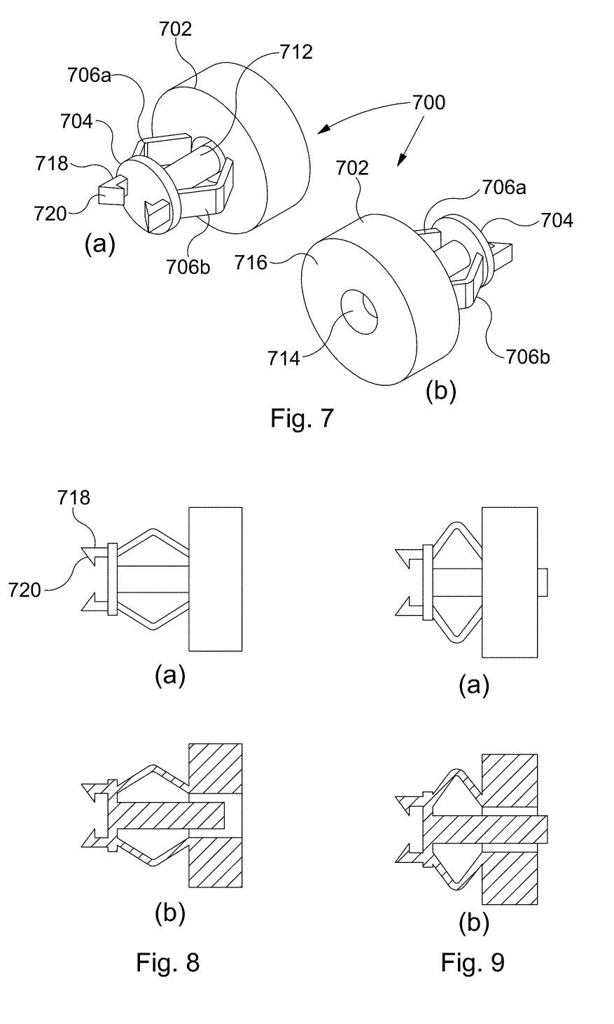

The coupling means 704 comprises one or more resiliently deformable members 718 comprises lugs 720. The deformable members 718 are configured to deform outwards as they pass over a head of a syringe plunger and then to snap back to engage the lugs 720 under the lip formed by the head of the syringe plunger. The exemplary pressure indicator of FIGS. 7-9 shows two resiliently deformable members, but there may be more than two, for example three, resiliently deformable members.

It is noted that the coupling means may take any of numerous forms and should not be limited to the examples shown in FIGS. 7-9 and described above.

FIGS. 5, 6, 8 and 9 show operation of the pressure indicators 400, 700. This is explained above in respect of the syringe plunger 100 and is not discussed again here.

The syringe plunger may be moulded from a single piece of plastics material. Alternatively, the syringe plunger may be provided as a kit of a number of separate parts. The kit may comprise the first portion, the second portion and the indicator. In exemplary kits, the indicator may be formed as part of the first portion or the second portion. The kit may also comprise the resiliently deformable member, which may be formed as part of the first portion and/or the second portion.

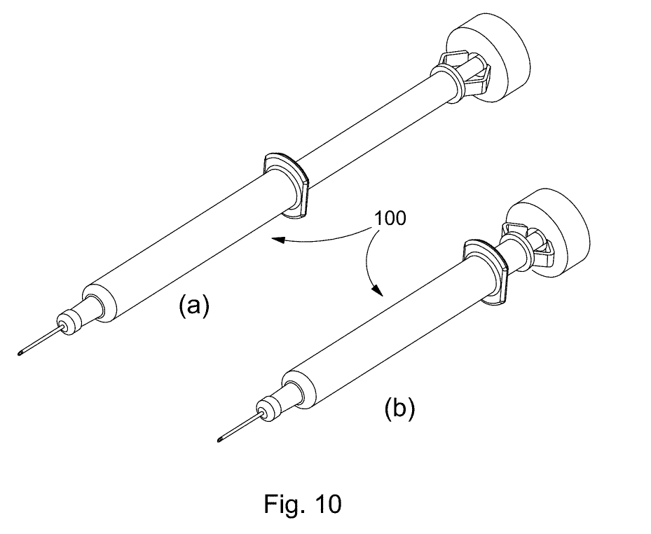

FIGS. 10a and 10b show perspective views of a syringe 1000 comprising a syringe plunger that is extended (FIG. 10a) and at least partially inserted (FIG. 10b) into a barrel of the syringe. The syringe comprises a syringe plunger as described herein. The syringe plunger may comprise a pressure indicator as a built in feature or may have a pressure indicator retro-fitted.

The skilled person will be able to envisage other embodiments of the invention without departing from the scope of the appended claims.

1. A syringe plunger for insertion within a barrel of a syringe for expelling the contents of the barrel from an open end thereof, the syringe plunger comprising:
a first portion;
a second portion moveable with respect to the first portion if a relative force between the first portion and the second portion exceeds a threshold; and
an indicator,
wherein translation of movement of the first portion relative to the second portion causes mechanical movement of the indicator.

2. A syringe plunger according to claim 1, wherein the first portion comprises a plunger head and the second portion comprises a plunger shaft.

3. A syringe plunger according to claim 1 or 2, further comprising a resiliently deformable member coupled to the first and second portions.

4. A syringe plunger according to claim 3, wherein the resiliently deformable member is configured to bias the first portion away from the second portion and/or bias the second portion away from the first portion.

5. A syringe plunger according to claim 4, wherein the resiliently deformable member is positioned between the first portion and the second portion and is configured to compress if the relative force between the first portion and the second portion exceeds the threshold.

6. A syringe plunger according to claim 3, wherein the resiliently deformable member is configured to bias the first portion towards the second portion and/or bias the second portion towards the first portion.

7. A syringe plunger according to claim 6, wherein the resiliently deformable member is positioned between the first portion and the second portion and is configured to expand if the relative force between the first portion and the second portion exceeds the threshold.

8. A syringe plunger according to any of claims 3 to 7, wherein the resiliently deformable member comprises one or more arms extending between the first portion and the second portion.

9. A syringe plunger according to claim 8, wherein the one or more arms are configured to flex with respect to the syringe plunger on movement of the first portion with respect to the second portion.

10. A syringe plunger according to any preceding claim, wherein the indicator is configured to provide a haptic indication to a user of a syringe.

11. A syringe plunger according to claim 10, wherein the indicator comprises an indicator rod configured to contact the user's skin after movement of the first portion with respect to the second portion.

12. A syringe plunger according to claim 11, wherein the indicator rod is fixed with respect to the second portion.

13. A syringe plunger according to claim 11 or 12, wherein the first portion comprises an aperture in a force application surface, and wherein the indicator rod is configured to pass through the aperture after movement of the first portion towards the second portion.

14. A pressure indicator for coupling to a syringe plunger, the pressure indicator comprising:
a first portion;
a second portion moveable with respect to the first portion if a relative force between the first portion and the second portion exceeds a threshold; and
an indicator,
wherein translation of movement of the first portion relative to the second portion causes mechanical movement of the indicator.

15. A pressure indicator according to claim 14, wherein the first portion comprises a plunger head and the second portion is configured to be directly coupled to a syringe plunger.

16. A pressure indicator according to claim 14 or 15, further comprising a resiliently deformable member coupled to the first and second portions.

17. A pressure indicator according to claim 16, wherein the resiliently deformable member is configured to bias the first portion away from the second portion and/or bias the second portion away from the first portion.

18. A pressure indicator according to claim 17, wherein the resiliently deformable member is positioned between the first portion and the second portion and is configured to compress if the relative force between the first portion and the second portion exceeds the threshold.

19. A pressure indicator according to claim 16, wherein the resiliently deformable member is configured to bias the first portion towards the second portion and/or bias the second portion towards the first portion.

20. A pressure indicator according to claim 19, wherein the resiliently deformable member is positioned between the first portion and the second portion and is configured to expand if the relative force between the first portion and the second portion exceeds the threshold.

21. A pressure indicator according to any of claims 16 to 20, wherein the resiliently deformable member comprises one or more arms extending between the first portion and the second portion.

22. A pressure indicator according to claim 21, wherein the one or more arms are configured to flex with respect to the syringe plunger on movement of the first portion with respect to the second portion.

23. A pressure indicator according to any of claims 14 to 22, wherein the indicator is configured to provide a haptic indication to a user of a syringe.

24. A pressure indicator according to claim 23, wherein the indicator comprises an indicator rod configured to contact the user's skin after movement of the first portion with respect to the second portion.

25. A pressure indicator according to claim 24, wherein the indicator rod is fixed with respect to the second portion.

26. A pressure indicator according to claim 24 or 25, wherein the first portion comprises an aperture in a force application surface, and wherein the indicator rod is configured to pass through the aperture after movement of the first portion with respect to the second portion.

27. A syringe plunger comprising the pressure indicator of any of claims 14 to 26.

28. A syringe comprising a barrel and a syringe plunger according to any of claims 1 to 13 and 27.

29. A kit of parts comprising:
a first portion of a syringe plunger;
a second portion of a syringe plunger; and
an indicator,
wherein the first portion is configured to be coupled to the second portion such that it is moveable with respect to the second portion if a relative force between the first portion and the second portion exceeds a threshold,
and wherein translation of movement of the first portion relative to the second portion causes mechanical movement of the indicator.

30. A kit of parts according to claim 29, wherein the indicator forms part of one of the first portion and the second portion.

31. A kit of parts according to claim 29 or 30, further comprising a resiliently deformable member coupled to the first and second portions.

32. A kit of parts according to claim 31, wherein the resiliently deformable member forms part of the first portion and/or the second portion.

33. A syringe plunger substantially as herein described with reference to the accompanying drawings.

34. A pressure indicator substantially as herein described with reference to the accompanying drawings.

35. A syringe substantially as herein described with reference to the accompanying drawings.
